(19) United States
(12) Patent Application Publication
(10) Pub. No.: US 2022/0076819 A1
(43) Pub. Date: Mar. 10, 2022

(54) Systems and Methods for Secure Transmission and Secure Storage of Medical Data from Standalone Medical Devices

(71) Applicant: TIENOVIX, LLC, Houston, TX (US)

(72) Inventors: Ramy Danial, Houston, TX (US); Kyle Q. Nguyen, League City, TX (US)

(73) Assignee: TIENOVIX, LLC, Houston, TX (US)

(21) Appl. No.: 17/013,533

(22) Filed: Sep. 4, 2020

(51) Int. Cl. G16H 40/67; G16H 10/60

(52) U.S. Cl. CPC G16H 40/67; G16H 10/60

(57) ABSTRACT

A system, comprising at least one medical device located at a medical facility, wherein each medical device is configured to transmit medical device data; a data extraction module configured to (a) receive the raw medical device data and (b) extract medical device data from the raw medical device data; medical data transfer module configured to (a) directly or indirectly receive the medical device data from the data extraction module and (b) securely transmit the data to a medical data cloud module; and the medical data cloud module, wherein the medical data cloud module is not located at the medical facility, and the medical data cloud module is configured to (a) receive the data securely transmitted by the medical data transfer module and (b) securely store the received data. Methods of securely transmitting and/or securely storing medical data by use of one or more components of the system.

### BACKGROUND OF THE INVENTION

The present disclosure relates to systems and methods for securely transmitting and securely storing medical data.

### SUMMARY

The present disclosure provides systems and methods for securely transmitting and securely storing medical data.

Although systems of the present invention may be described for particular medical devices and medical device systems, persons of skill in the art having the benefit of the present disclosure will appreciate that these systems may be used in connection with other medical devices not specifically noted herein. Further, it will also be appreciated that systems according to the present invention not involving medical applications are also within the scope of the present invention. For example, systems of the present invention may be used in many industrial or commercial settings to train users to operate may different kinds of equipment, including heavy machinery as well as many types of precision instruments, tools, or devices. Accordingly, the particular embodiments disclosed above are illustrative only, as the invention may be modified and practiced in different but equivalent manners apparent to those skilled in the art having the benefit of the teachings herein. Examples, where provided, are all intended to be non-limiting. Furthermore, exemplary details of construction or design herein shown are not intended to limit or preclude other designs achieving the same function. The particular embodiments disclosed above may be altered or modified and all such variations are considered within the scope and spirit of the invention, which are limited only by the scope of the claims.

In one embodiment, the present disclosure relates to a system, comprising at least one medical device located at a medical facility, wherein each medical device is configured to transmit raw medical device data; a data extraction module configured to (a) receive the raw medical device data and (b) extract medical device data from the raw medical device data; medical data transfer module configured to (a) directly or indirectly receive the medical device data from the data extraction module and (b) securely transmit the data to a medical data cloud module; and the medical data cloud module, wherein the medical data cloud module is not located at the medical facility, and the medical data cloud module is configured to (a) receive the data securely transmitted by the medical data transfer module and (b) securely store the received data.

In one embodiment, the present disclosure relates to a method, comprising transmitting raw medical device data by at least one medical device located at a medical facility; extracting medical device data from the raw medical device data by a data extraction module; transmitting, securely, the data to a medical data cloud module; receiving the securely transmitted data by a medical data cloud module, wherein the medical data cloud module is not located at the medical facility; and storing, securely, the received data by the medical data cloud module.

In one embodiment, the present disclosure relates to a method, comprising detecting an initiation of a medical device; establishing a communication protocol between the medical device and a data extraction module; establishing a control protocol between the medical device and a virtual machine; establishing a secure communication link from the medical device through the data routing module to a medical data cloud module; transmitting medical data from the medical device to the medical data cloud module over the secure communication link; and storing the medical data by the medical data cloud module.

### BRIEF DESCRIPTION OF THE DRAWINGS

FIG. 1A is a first block diagram view of a medical device data network, in accordance with embodiments herein.

FIG. 1B is a second block diagram view of a medical device data network, in accordance with embodiments herein.

FIG. 1C is a stylized, block diagram depiction of the data extraction module 128, in accordance with some embodiments herein.

FIG. 1D is a flowchart representation (180) of an operation performed by the extraction module 128, in accordance with embodiments herein.

FIG. 2 is a block diagram various aspects of the medical device data network of FIG. 1 in more detail, in accordance with embodiments herein.

FIG. 3 is a block diagram of a medical data controller, in accordance with embodiments herein.

FIG. 4 is a block diagram of a first medical device controller, in accordance with embodiments herein.

FIG. 5 is a block diagram of a second medical device controller, in accordance with embodiments herein.

FIG. 6 is a block diagram depicting a first data processing module, in accordance with embodiments herein.

FIG. 7 is a flowchart of a first method, in accordance with embodiments herein.

FIG. 8 is a flowchart of a first method, in accordance with embodiments herein.

### DESCRIPTION

Exemplary embodiments are illustrated in referenced figures of the drawings. The embodiments disclosed herein are considered illustrative rather than restrictive. No limitation on the scope of the technology and on the claims that follow is to be imputed to the examples shown in the drawings and discussed here.

In one embodiment, the present disclosure relates to a system, comprising at least one medical device located at a medical facility, wherein each medical device is configured to transmit medical device data; a medical data transfer module configured to (a) directly or indirectly receive the medical device data transmitted by the at least one medical device and (b) securely transmit the data to a medical data cloud module; and the medical data cloud module, wherein the medical data cloud module is not located at the medical facility, and the medical data cloud module is configured to (a) receive the data securely transmitted by the medical data transfer module and (b) securely store the received data.

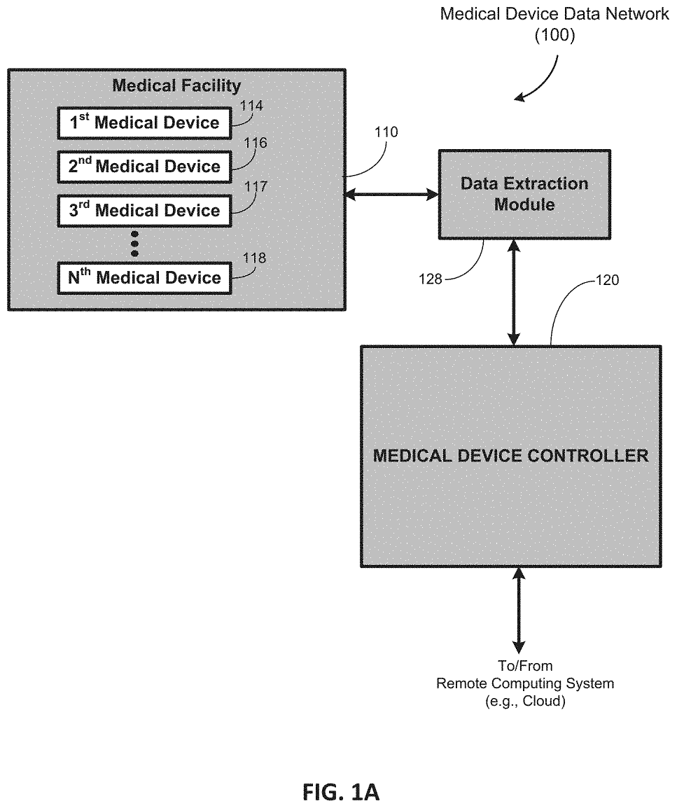

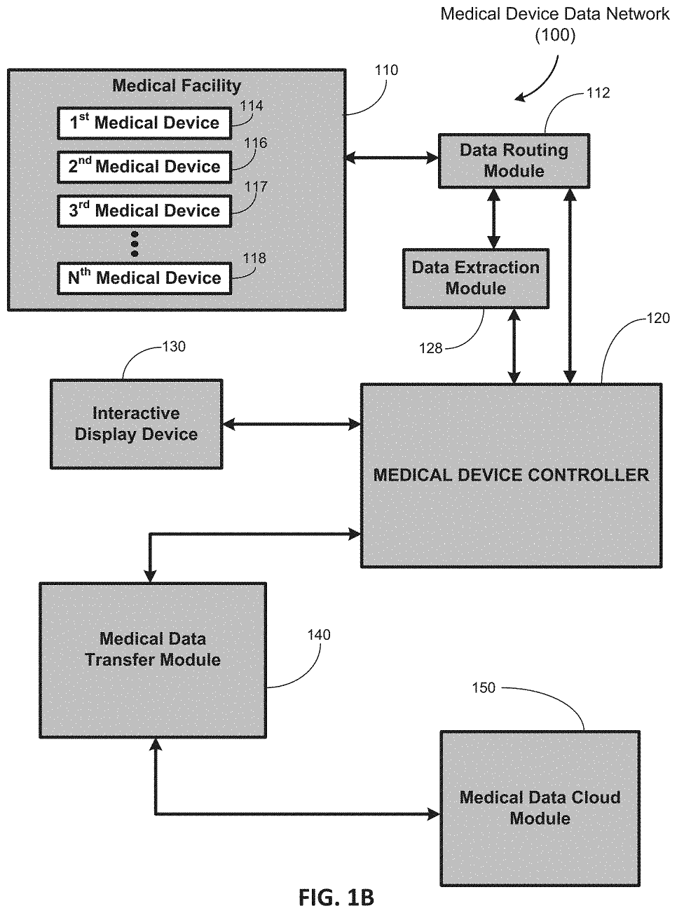

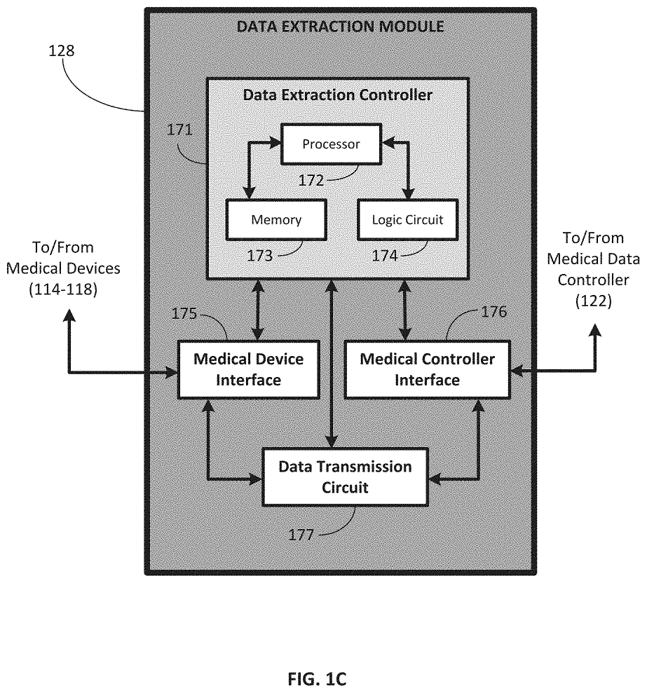

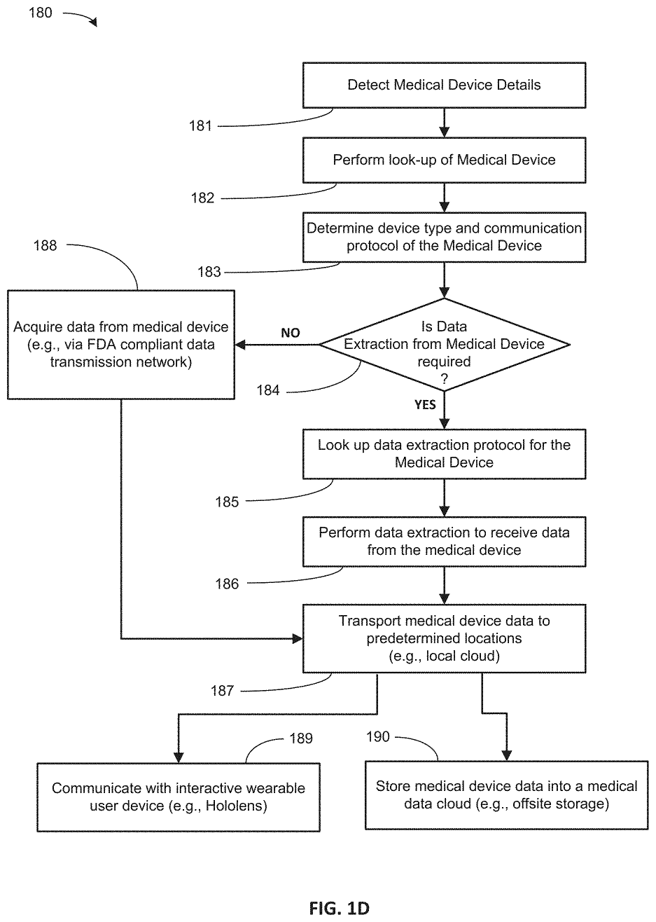

FIG. 1A and FIG. 1B present stylized, block diagram depictions of an exemplary medical device data network 100, in accordance with one or more embodiments herein. At a medical facility 110, which may be a hospital, a clinic, a teaching hospital, a medical school, a nursing home, an assisted living facility, an emergency deployment (e.g., an ambulance, a battlefield medicine site, etc.), or the like, are located at least one medical device 114-118. In the depicted embodiment, at the medical facility 110 are located a first medical device 114, a second medical device 116, a third medical device 117, up to an N^{th} medical device 118. In one embodiment, two or more of the different medical devices 114-118 may be multiple instances of the same type of medical device, e.g., two or more ultrasound machines, cardiac data monitors, etc., . . . , while in other embodiments, each of the different medical devices 114-118 may be of a different type than the others, or may be a combination thereof. One or more of the medical devices 114-118 are not capable of being connected to the Internet. At least one of the medical devices 114-118 is a standalone medical device, or a legacy medical device. “Standalone medical device” or “legacy medical device” generally refer to medical devices that are not connected or are not capable of being connected, to the Internet.

The medical device data network 100, as shown in FIG. 1A, comprises a data extraction module 128. The data extraction module 128, as shown, is configured to receive raw medical device data gathered by the medical devices 114-118, and to extract medical data from the raw medical device data in a form suitable for further operations by the medical device data network 100, such as secure transmission to medical data cloud module 150 and/or secure storage of the medical data by the medical data cloud module 150. “Extraction” comprises any of a number of operations, including, but not limited to, receiving data, buffering data, signal-to-noise processing, band pass filtering, digital signal processing, analog-to-digital conversion, or stream editing operations (e.g., converting straight quotes to curly quotes or vice versa, converting MS-DOS line ending characters to Unix line ending characters or vice versa, etc.), among others that will be apparent to the person of ordinary skill in the art having the benefit of the present disclosure.

Extraction of data performed by the data extraction module 128 may include interfacing with one or more medical devices 114-118 in a manner to receive medical data and/or metadata associated with an operation of the medical device. This data exchange may be performed by wired connection, via an internal network of a facility, and/or by wireless connection.

The data extraction module 128 may detect or may receive information as to what type of medical device with which it is interfacing. Based on this information, the data extraction module 128 may perform a data conversion directed to the specific type of medical device with which it is interfacing. The type of data conversion to be performed may be based on information stored within the data extraction module 128, or in an alternative embodiment, may be based on a look up process performed by the data extraction module 128.

In some embodiments, the data extraction module 128 may comprise its own processor, memory, control circuits, and interface circuits for receiving, buffering, processing, converting, and/or transmitting medical data received from one or more of the medical devices 114-118. In other embodiments, some or significant amount of the controlling and look up functions performed by the data extraction module 128 may be based on using the processing resources and/or memory resources of an external module, such as the medical device controller 120. A more detailed illustration of the Data Extraction Module is provided in FIG. 1C and accompanying description below.

FIG. 1C illustrates a stylized, block diagram depiction of the data extraction module 128, in accordance with some embodiments herein. The data extraction controller 128 comprises a data extraction controller 171. The data extraction controller 171 is capable of controlling the operations of the data extraction module 128. The data extraction controller 171 may comprise a processor 172 and a logic circuit 174 that are capable of directing the operations of the data extraction module 128. The data extraction controller 171 and/or the logic circuit 174 may be comprised of hardware, software, and/or firmware components. For example, the logic circuit may be a FPGA (field programmable gate array) or an ASIC (application specific integrated circuit).

The data extraction controller 171 may also comprise a memory 173 that may store various information required for the operation of the data extraction module 128. For example, the memory 173 may store program information, look-up information regarding various medical devices, operation mode data, processor operation program, etc.

The data extraction module 128 may also comprise a medical device interface 175. The medical device interface 175 facilitates the extraction of data (e.g., medical device output data, metadata, patient data, etc.) from one or more medical devices 114-118. The medical device interface provides a conduit for the data extraction controller 171 to control data flow to and from the medical devices 114-118.

The data extraction module 128 may also comprise a medical controller interface 176. The medical controller interface 176 provides for communications between the data extraction controller 171 and the medical data controller 122.

The data extraction module 128 may further comprise a data transmission circuit 177. The data transmission circuit 177 provides for transmission of data between various elements of the data extraction module 128, such as between two or more of the data extraction controller 171, the medical device interface 175, and/or the medical controller interface 176.

One or more portions of the data extraction module 128 may be hardware modules, software modules, firmware modules, and/or a combination thereof. Firmware modules may include programmable devices such as field programmable gate arrays (FPGAs) or application-specific integrated circuits (ASICs)

Turning now to FIG. 1D, a flowchart representation (180) of an operation performed by the extraction module 128, in accordance with embodiments herein, is illustrated. Upon activation of a medical device, a determination may be made regarding the details of the medical device (at 181). These details may include information as to the type of medical device, the generation of the hardware and/or software of the medical device, the communication capabilities and protocols of the medical devices, etc.

Upon receiving the details of the medical device, the data extraction module 128 may perform a look-up function to acquire necessary communications and other technical details of the medical device (at 182). Based on this look-up and/or the medical device details, the data extraction module 128 determines the medical device type and communication protocol required (at 183).

Based on this inquiry, a determination is made whether data extraction from the medical devices is required (at 184). For example, if the medical device is type of device that is not readily interfaced with the Internet, or if the medical devices is a legacy device, a data extraction process may be required in order to acquire data from the medical device.

If a determination is made that the medical that data extraction is not required for the medical device, a process for acquiring data from medical device may be performed (at 188). In one embodiment, this acquisition of data, which may include medical data of a patient, metadata, etc., may be performed via a data transmission network that is compliant with FDA medical data transmission requirements.

If a determination is made that data extraction is not required, a look up of the data extraction protocol for the medical device may be performed (at 185). Using protocol, the data extraction module 128 may perform a data extraction to receive data from the medical device (at 186). This data may include patient medical data as well as various metadata. Whether data extraction is performed or not, a process for transporting medical data to predetermined location is performed (at 187). This process may involve storing the medical device data into a local cloud, such as a local cloud network of a medical facility, such as a hospital, a nursing home, etc.

The medical data may also be sent to an interactive wearable device, such as a Hololens, for further interaction between a patient and the medical device (at 189). Further, the medical device data may be stored into a cloud, such as a medical data cloud that is in compliance with FDA regulations for medical data storage (at 190). This storage may be located in a site that is remote from the facility in which the medical device resides.

Turning back to FIG. 1B, the medical device data network 100 shown in FIG. 1B also comprises a data routing module 112. Generally, the data routing module 112 is configured to send data extracted by the data extraction module 128 to, and/or receive data from, a medical device controller 120. The data routing module 112 may be configured to provide capabilities for transmitting and receiving data in one or more data protocols.

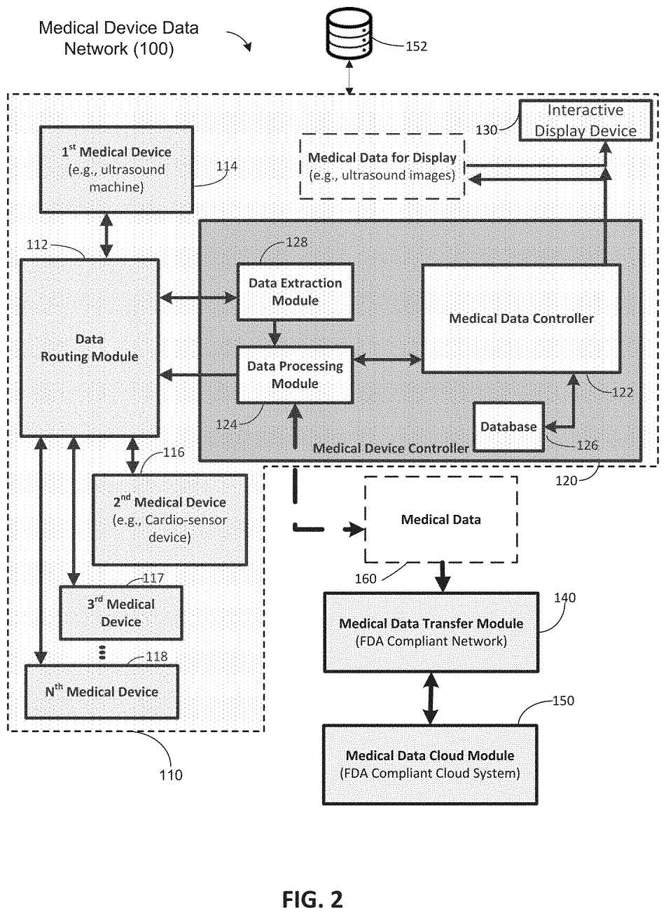

The medical device data network 100 also comprises a medical device controller 120. The medical device controller 120 is configured to control the 1^{st} through N^{th} medical devices 114-118, and/or send/receive data to/from a cloud. (as shown in FIG. 1A). Sending and receiving data from the cloud may comprise sending and/or receiving data to/from a medical data transfer module 140 and ultimately to storage in a medical data cloud module 150 (FIG. 1B). As shown in FIG. 1B, the medical device controller 120 may also send and/or receive data to/from an interactive display device 130, as will be discussed in more detail below in the context of FIG. 2-FIG. 6.

The medical device data network 100 may further comprise a medical data transfer module 140. The medical data transfer module 140 is configured to send and receive data from the medical device controller 120 and medical data cloud module 150, as will be discussed in more detail below in the context of FIG. 2-FIG. 6.

The medical device data network 100 may also comprise a medical data cloud module 150. The medical data cloud module 150 is configured to send and receive data from the medical data cloud module 150 and is also configured to securely store medical data send to it through medical data transfer module 140, as will be discussed in more detail below in the context of FIG. 2-FIG. 6.

In embodiments, the medical device data network 100 additionally comprises an interactive display device 130. The interactive display device 130 is configured to send and receive data from the medical device controller 120. This data may include control data, status data, medical data, etc. Control data may include one or more digital and/or analog signals that prompt one or more actions to be performed by a medical device. Status data may include one or more digital and/or analog signals that is indicative of one or more status of a medical devices, such as mode data, power status, operation status, metadata, etc. Medical data may include various types of information regarding a patient, patient condition, medical results, scan results (e.g., ultrasound data, CT scan data, MRI data, etc.), heart-related data, endocrine data, respiratory data, neurological data, and/or the like.

The interactive display device 130 is also configured to display data through an interactive display to a user. The interactive display device 130 may display medical data gathered by a medical device 114-118 The interactive display device 130 may display such medical data to the user when the user is performing a medical procedure using the medical device 114 et seq, as will be discussed in more detail below in the context of FIG. 2-FIG. 6. In other words, the interactive display device 130 may display real-time images, graphs, or other graphical elements to the user of the medical device 114-118. The interactive display device 130 also provides for controlling the medical devices 114 et seq. and receiving instructions and/or other guidance to be conveyed to the user of the interactive display device 130.

In one embodiment, the interactive display device 130 may be a HoloLens® or a HoloLens2® (Microsoft Corp., Redmond, Wash.), among other known virtual reality (VR), augmented reality (AR), mixed reality (MR), and/or extended reality (XR) devices.

One or more components of the data extraction module 128, the data routing module 112, the medical device controller 120, the interactive display device 130, the medical data transfer module 140, and the medical data cloud module 150 may be hardware components, software components, and/or firmware components. Particular structural features required for the data routing module 112, the medical device controller 120, the interactive display device 130, the medical data transfer module 140, and the medical data cloud module 150 to perform their configured functions will be known to the person of ordinary skill in the art having benefit of the present disclosure, and need not be described in detail.

FIG. 2 depicts a more detailed, stylized depiction of medical device data network 100 of FIG. 1 in accordance with one or more embodiments herein. The medical device controller 120 may interact with a plurality of medical devices, e.g., the 1^{st} through N^{th} medical devices 114, et seq. In one exemplary embodiment, the first medical device 114 is an ultrasound imaging device; the second medical device 116 is a cardiosensor device, such as an electrocardiogram (EKG) device, a heart rate monitor, a heart rate variability (HRV) monitor, and/or a pulse oximeter, among others.

The third medical device 117 through the N^{th} medical device 118 may be any known medical devices. In this embodiment, the medical devices 114-118 may be legacy devices that generate raw medical device data, wherein the raw medical device data is not directly suitable for further operations, such as secure transmission and/or secure storage.

FIG. 2 shows one embodiment of connections between the data routing module 112 and the medical devices 114-118. In this embodiment, the data routing module 112 is configured to (a) receive the medical device data transmitted by each medical device.

In addition, in embodiments, the data routing module 112 may be configured to send data received from the medical device controller 120 to the medical devices 114-118. The data transmitted via the data routing module 112 may be control data, status data, medical data, etc. For example, the data sent by the data routing module 112 to the medical devices 114-118 may be commands generated by a medical data controller 122 for control of the medical devices 114-118. The commands from the medical data controller 122 to the data routing module 112 may be relayed via a data processing module 124 if desired.

The data routing module 112 is configured to (b) directly or indirectly transmit the data received from each medical device to the medical data transfer module. Specifically, the data routing module 112 of FIG. 2 is configured to directly transmit the data to the data extraction module 128, from which further processing leads to transmission of the data to the medical data transfer module 140 via data processing module 124 of the medical device controller 120. In other embodiments (not shown), the data medical device data network 100 may omit the data processing module 124 and/or the medical data transfer module 140.

In the depicted embodiment of FIG. 2, the medical device controller 120 comprises a data extraction module 128, a data processing module 124, medical data controller 122, and a database 126. Each of these components will now be described in more detail.

The data extraction module 128, as shown, is configured to receive raw medical device data gathered by the medical devices 114-118, and to extract medical data from the raw medical device data in a form suitable for further operations by the medical device data network 100, such as secure transmission to medical data cloud module 150 and/or secure storage of the medical data by the medical data cloud module 150. “Extraction” comprises any of a number of operations, including, but not limited to, signal-to-noise processing, band pass filtering, digital signal processing, analog-to-digital conversion, or stream editing operations (e.g., converting straight quotes to curly quotes or vice versa, converting MS-DOS line ending characters to Unix line ending characters or vice versa, etc.), among others that will be apparent to the person of ordinary skill in the art having the benefit of the present disclosure.

The data processing module 124, as shown, is configured to send and receive data, such as medical data gathered by the medical devices 114-118 and extracted from raw medical device data by the data extraction module 128, directly or indirectly to the medical data cloud module 150, such as through a medical data transfer module 140. In order to comply with relevant medical data security regulations promulgated by industry standards, or government authorities such as the United States Food and Drug Administration (FDA) and comparable authorities in other sovereign jurisdictions, the transmission of medical data 160 from the data processing module 124 to the medical data transfer module 140 and the transmission of medical data 160 from the medical data transfer module 140 to the medical data cloud module 150 must be performed under certain security protocol(s) relevant to corresponding regulations/laws. Accordingly, the data processing module 124 may be configured to process the medical data generated by the medical devices 114-118, which is not typically secure enough to comply with relevant regulations, to yield secure medical data, and this secure medical data is then sent directly or indirectly to the medical device controller 120. The secure medical data may have undergone encryption or other comparable processing to be made secure. Alternatively, or in addition, the medical data 160 may be transmitted by a secure pathway, e.g., by virtual private network (VPN), SSH, blockchain, or other routes by which cleartext data may be transmitted in a secure manner.

In embodiments, wherein the medical data transfer module 140 is located at the medical location 110 or is under the control of the same entity as the medical location 110, the medical data transfer module 140 may securely store the medical data alternatively to or in addition to securely transmitting it to the medical data cloud module 150. The medical data transfer module 140 in this embodiment may perform one or more data conversions required for secure transmission across networks outside of the entity's control. In this embodiment, the medical data transfer module 140 may be considered an intermediate cloud.

Accordingly, the medical device controller 120 may be configured to (a) to control one or more operations of or more medical devices 114-118; (b) directly or indirectly receive the medical device data transmitted by each medical device, and (c) transmit the data received from each medical device to the medical data transfer module.

The medical device controller 120 also comprises a medical data controller 122. The medical data controller 122 may be configured, by virtue of particular structural features which will be known to the person of ordinary skill in the art, to perform one or more operations.

The medical data controller 122 may be configured to perform the running of one or more virtual machines, each of which may be used to control one or more of the medical devices 114-118. Such virtual machines will be described in more detail below.

Alternatively, or in addition, the medical data controller 122 may be configured to interface with the interactive display device 130. In one embodiment, the interface with the interactive display device 130 may comprise receiving medical data gathered from the medical devices 114-118 and passed through the data routing module 112, the data extraction module 128, and the data processing module 124 and providing that medical data to the interactive display device 130. The medical data received by the medical data controller 122 may be in a format suitable for display, but if not, the medical data controller 122 may perform one or more data operations thereon to render the data suitable for display.

In further embodiments, the medical data controller 122 may also be configured to serve instructions for performing a medical procedure to a user using the interactive display device 130 and at least one medical device 114-118. In an embodiment, the instructions comprise at least one augmented reality element, such as a text element, a video element, an icon, a sound, a narration, or a haptic element, among others known to the person of ordinary skill in the art having the benefit of the present disclosure.

In other words, the medical device controller 120 may be configured to (c) control the at least one medical device 114-118, (d) provide medical data in a graphical format to the interactive display device 130, (e) provide instructions for a medical procedure making use of the at least one medical device 114-118 to the interactive display device 130, or (f) two or more of (c)-(e).

The medical device controller 120 may also comprise a database 126. In alternative embodiments, the database 126 may be located outside of the medical device controller 120. The database 126 may be a centralized database, a distributed database, personal database, an end-user database, a commercial database, a standard Query Language (SQL) database, a NoSQL database, a graph database, an operational database, a relational database, a cloud database, an object-oriented database, or any combination thereof. The database 126 may contain a procedure library accessible by the medical data controller 122 to generate instructions for a medical procedure making use of the at least one medical device 114-118 to the interactive display device 130 and/or authorization data to allow the medical data controller 122 to control the at least one medical device 114-118.

In the particular embodiment shown in FIG. 2, the medical devices 114-118, the data routing module 112, the data extraction module 128, the medical device controller 120, and the interactive display device 130 are shown as being at the medical location 110. The precise boundary of the medical location 110 may be varied as a routine matter. It may be desirable to include the entire medical device controller 120 at the medical location 110, thereby reducing the need to transfer medical data to remote locations requiring greater security during transfer and/or storage than may prevail if the entire medical device controller 120 is located at the medical location 110 and may make use of the medical location 110's preexisting intranet security infrastructure.

There may also be advantageous, of cost, size, power consumption, simplified support, etc. to incorporate a network switching appliance and a routing appliance in the medical device controller 120. A combined switching/routing appliance may be used. Any switching and/or routing appliance used in the medical device controller 120 or elsewhere in the medical device data network 100 may be deployed as a serviceable and/or replaceable physical unit, which may provide improved cybersecurity at the cost of larger unit size, or may be embedded in the processor module 610 to be described below, running the risk of reduced cybersecurity when the processor module 610 reaches end-of-life.

In addition to the medical data cloud module 150, FIG. 2 depicts an embodiment in which the medical device data network 100 comprises a medical collaboration cloud module 152. The medical collaboration cloud module 152 may be used by one or more users of one or more devices of the medical device data network 100 to store, check in, check out, read, write, watch, edit, etc. one or more data types or files relating to the medical device data network 100 and/or the medical facility 110. For example, the medical collaboration cloud module 152 may allow streaming, storage, viewing, annotating, assigning for follow up, etc. of the medical data generated by one or more of the medical devices 114-118, such as the medical data for display generated by the medical data controller 122 and/or inputs and outputs of the interactive display device 130; records of patients at the medical facility 110; word processing documents, presentations, spreadsheets, and other documents generated by an office suite, such as Microsoft Office or Google Docs, among others; operational data relating to one or more of the medical devices 114-118; etc. Desirably, this data is transmitted to and stored by the medical collaboration cloud module 152 in compliance with all relevant data security regulations and/or laws.

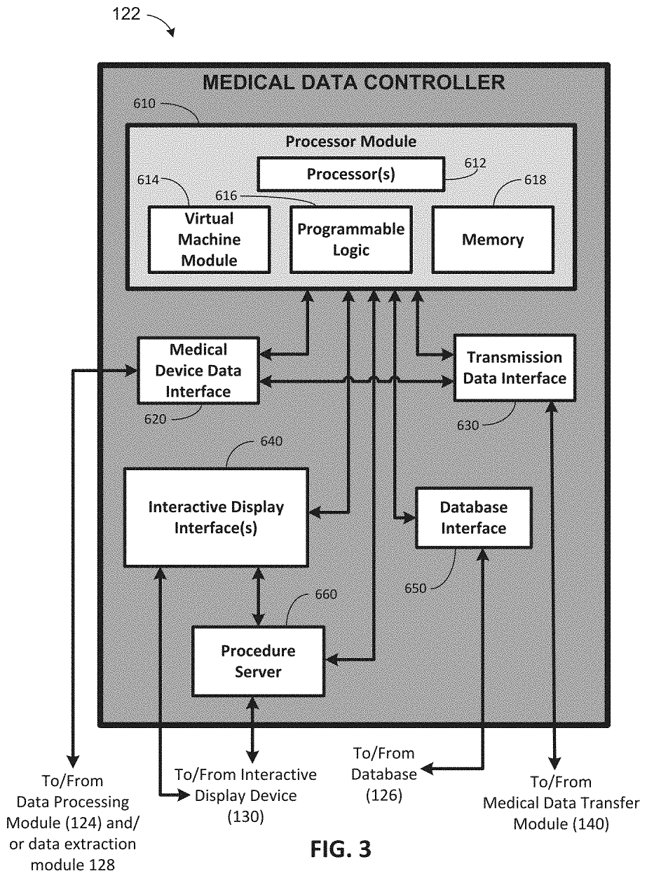

FIG. 3 shows a stylized, block diagram depiction of the medical data controller 122 in more detail, in accordance with embodiments herein. The medical data controller 122 comprises a processor module 610. The processor module 610 may comprise one or more processor(s) 612. The processor(s) 612 may be separate CPUs, multiple cores of a single CPU, microcontrollers, FPGA-based processors, or other variations in hardware that may be routinely used by the person of ordinary skill in the art having the benefit of the present disclosure.

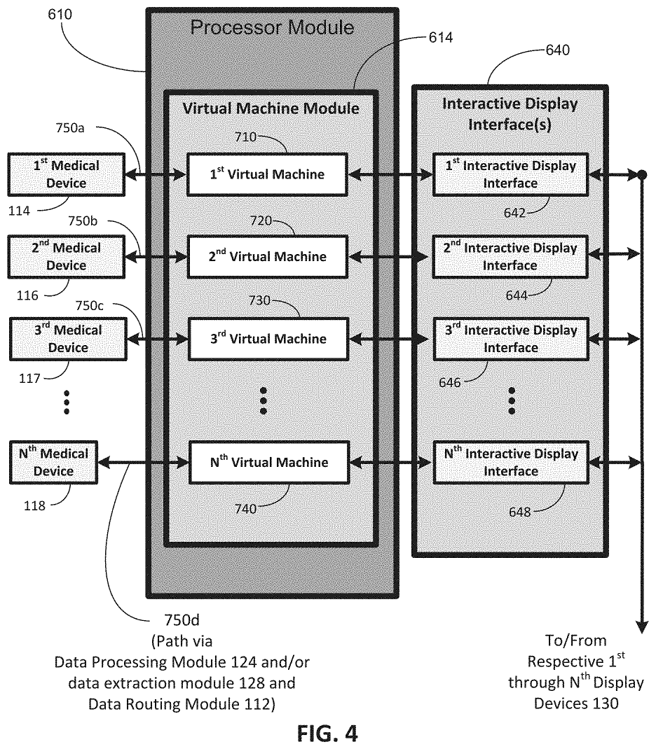

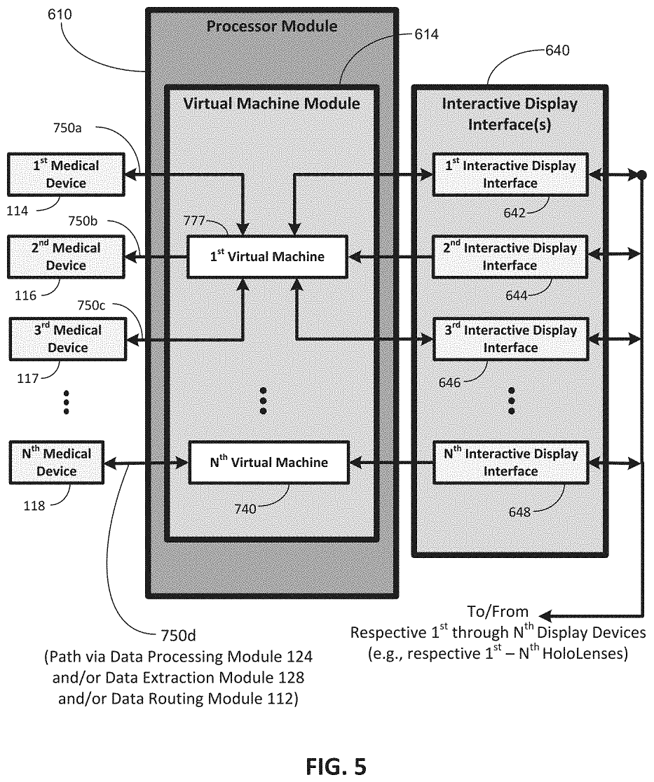

The processor module 612 may also comprise a virtual machine module 614, which will be discussed in more detail with reference to FIG. 4 and FIG. 5, below. The processor module 610 may also comprise programmable logic 616, such as a hardware chip and/or software and/or firmware, such as a FPGA (field-programmable gate array) or a ASIC (application-specific integrated chip) device. The programmable logic 616 may be configured to perform control operations and/or logical operations on data received from one or more other locations in the medical data controller 122, the medical device controller 120, and/or the medical device data network 100.

The processor module 610 may also comprise a memory 618, such as RAM and/or a readable and writable fixed storage device (e.g., a hard disk, a solid state drive, etc.). In an exemplary embodiment, the processor module 610 may be the motherboard and connected hardware (e.g., CPU, RAM, etc.) of a custom-built or prebuilt computer, onto which necessary software may be installed with a reduced need for installation-specific customization of the processor module 610.

The processor module 610 may interface bidirectionally, such as through an I/O bus, with one or more other modules 620-660 of the medical data controller 122. The other modules 620-660 may each be hardware, software, and/or firmware. It should be borne in mind that each module 620-660 is depicted conceptually and need not be instantiated as a particular separate physical unit. Though depicted as separate from one another and from processor module 610 in FIG. 3, the person of ordinary skill in the art will understand that part or all of any or all of the modules 620-660 may be located in the processor module 610, i.e., one or more operations performed by a module 620-660 may be performed by the processor 612.

As depicted in FIG. 3, the medical data controller 122 may comprise a medical device data interface 620. The medical device data interface 620 may transmit data to or receive data from the data processing module 124 and/or the data extraction module 128. As an example only, the medical device data interface 620 may receive data originating from medical device(s) 114-118 and provide it to one or more other components of the medical data controller 122. The medical device data interface 620 may also send data, such as control data and/or metadata that may be generated by the virtual machine module 614, to the medical device(s) 114-118 via the data processing module 124. The medical device data interface 620 may also provide medical data that is to be securely transmitted and/or securely stored to a transmission data interface 630.

The medical data controller 122 may comprise a transmission data interface 630. The transmission data interface 630 may transmit data to or receive data from the medical data transfer module 140, possibly by way of data processing module 124. In one embodiment, the transmission data interface 630 may perform necessary encrypting, checksum generating, signing, or other securing operations on the data to be transmitted to the medical data transfer module 140, and/or necessary decrypting, checksum verifying, signature verifying, etc. on data received from the medical data transfer module 140.

The medical data controller 122 may also comprise an interactive display interface 640. The interactive display interface 640 may receive medical data and/or procedure instructions from the processor module 610 and transmit the medical data and/or procedure instructions to the interactive display device 130. The interactive display interface 640 may, alternatively or in addition, may receive procedure instructions, guidance data, and/or related data directly from a procedure server 660. Also, the interactive display interface 640 may receive data from the interactive display device 130 relating to the use of the interactive display device 130 in a medical procedure making use of the medical device(s) 114-118. For example, the interactive display interface 640 may receive data relating to the relative or absolute location, speed of motion, vector of motion, or the like of the interactive display device 130, the medical device 114-118, and/or a user making use of the interactive display device 130 and the medical device 114-118, and transfer this data to the processor module 610 for use in generating procedure instructions that take into account the location, speed of motion, vector of motion, etc. information.

The medical data controller 122 may, as shown, comprise a procedure server 660. The procedure server 660 may generate procedure instructions and/or provide libraries, modules, baseline instructions, template instructions, etc. that the processor module 610 may use to generate procedure instructions. The procedure server 660 may communicate directly with the interactive display device 130, with the interactive display interface 640, with the processor module 610, or two or more thereof.

The medical data controller 122 may also comprise a database interface 650. The database interface 650 may permit communication between the processor module 610 and the database 126. For example, the database interface 650 may comprise an API for allowing programmable logic 616 written in a first programming and/or scripting language to send queries to and retrieve information from a second programming and/or scripting language framework controlling access to the database 126. The communications between the database 126 and the processor module 610 may allow the processor module 610 to access information from the database 126 for use in generating procedure instructions and/or controlling medical device(s) 114-118 from the virtual machine module 614.

FIG. 4 shows, in block diagram form, one embodiment of the virtual machine module 614. The virtual machine module 614 may be software instantiated by the operating system of the processor 612 and configured to emulate one or more instances of a computer running the same or a different operating system, although other implementations may be possible. The virtual machine module 614 may comprise one or more virtual machines, such as the first through N^{th} virtual machines 710-740 shown. In the depicted embodiment of FIG. 4, a first virtual machine 710 receives data from the first medical device 114 and sends instructions relating to the control of the first medical device 114. The first virtual machine 710 may also send and receive data from a first interactive display interface 642 of the interactive display interface 640. The first interactive display interface 642 may provide graphical medical data, procedure instructions, metadata, etc. to a user of the first medical device 114 and the or a corresponding interactive display device 130.

Similarly, the virtual machine module 614 may comprise a second virtual machine 720, a third virtual machine 730, up to an N^{th} virtual machine 740. Each of the virtual machines 720-740, similarly to the first virtual machine 710, may receive data from and send instructions relating to the control of a corresponding medical device 116-118. The virtual machines 720-740 may also send and receive data from a corresponding interactive display interface 644-648 of the interactive display interface 640, which may provide graphical medical data, procedure instructions, etc. to a user of the corresponding medical device 116-118 and an interactive display device 130.

The term “virtual machine” as used herein encompasses both emulated standalone computing devices containing operating systems, I/O device interfaces, etc. as well as containers, sandboxes, snaps, and other containerized instantiations of one or more programs and their dependencies, e.g., libraries and the like, operating under the operating system and related components of the processor module 610. Virtual machines and/or containerization may provide greater security than bare metal embodiments of the software and systems. Virtual machines may also refer to a firmware processor residing in an FPGA or an ASIC.

The use of virtual machines and/or containers provides flexibility to deployments of the medical device data network 100. For deployments involving relative low numbers of medical devices 114-118 and interactive display devices 130, a “virtual machine” may be an instance of software running on the processor 612. In larger deployments with relatively small data requirements, a “virtual machine” may be a containerized instance of software. In even larger deployments, and/or those with complex image processing and/or machine learning/AI backend activity, a “virtual machine” may be an emulated standalone computing device with access to a dedicated share of the physical resources of the processor module 610.

FIG. 5 presents an alternative embodiment with many similarities to that shown in FIG. 4. A significant difference between FIG. 4 and FIG. 5 is that the first virtual machine 777 of FIG. 5 may receive data from and send data to a plurality of medical devices 114-118 and a plurality of interactive display interfaces 642-648. In this scenario, the first virtual machine 777 may run one or more instances of a given program, and each program instance may interact with one or more medical devices 114-118 and interactive display interfaces 642-648. That is, a single virtual machine may interact with multiple interactive display interfaces. Thus, in the embodiment shown in FIG. 5, the interactive display interface(s) 640 may comprise M interface display interfaces, wherein M is an integer that is less than the integer N.

The virtual machine module 614 of FIG. 5 shows a first virtual machine 777 as described immediately above and an N^{th} virtual machine 740 as described in relation to FIG. 4. In other embodiments, not shown, a virtual machine module 614 may include only one or virtual machines like the first virtual machine 777, each interfacing with more than one medical devices and/or more than one interactive display interface. Such variations in how virtual machine(s) are implemented may depend on the complexity of the medical devices 114-118, the complexity of the data generated by and/or transmitted to the medical devices 114-118, the complexity of the interactive display interfaces 640-648, the complexity of the data generated by and/or transmitted to the interactive display interfaces 640-648, the properties of the emulated operating system and/or software of a virtual machine, the properties of the virtual machine module 614, the properties of the processor module 610, and/or others that will occur to the person of ordinary skill in the art having the benefit of the present disclosure.

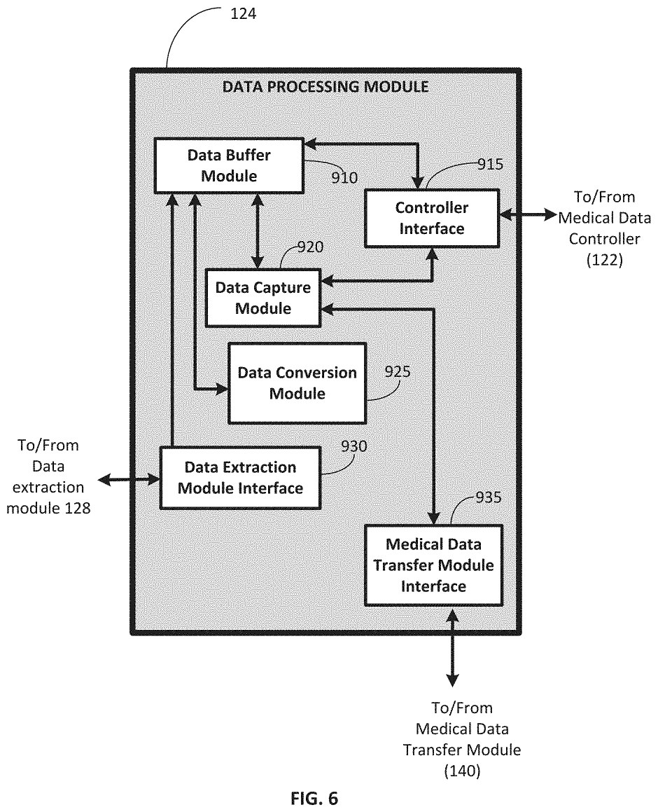

Turning to FIG. 6, a stylized, block diagram depiction of the data processing module 124 in greater conceptual detail. The data processing module 124 comprises a data buffer module 910. The data buffer module 910 may receive, send, and/or temporarily buffer or store data to/from the medical devices 114-118 (via data routing module 112), the medical data controller 122 (via a controller interface 915) and other components of the data processing module 124. The data buffer module 910 may comprise a FIFO (first-in-first-out) device to allow for desired syncing of data. In some embodiments, the data buffer module 910 may comprise a PLL (phase locked loop) to help sync data flow to desired clock signal(s).

The data processing module 124 may comprise a data extraction module interface 930. The data extraction module interface 930 may interface with the data extraction module 128 shown in FIG. 2 to receive extracted medical device data, and may provide the extracted data to the data buffer module 910 for transfer to other elements of the data processing module 124 and/or the medical device data network 100. The data extraction module interface 930 may comprise any suitable hardware, software, and/or firmware, as will be a matter of routine experimentation for the person of ordinary skill in the art having the benefit of the present disclosure.

The data processing module 124 may further comprise a data conversion module 925. The data conversion module 925 may operate on data received from the data extraction module interface 930 by the data buffer module 910 to convert it to a format suitable for further operations, including but not necessarily limited to secure transmission to and secure storage by the medical data cloud module 150. In one embodiment, the data conversion module 925 encrypts, signs, compresses, generates a check sum, etc. the extracted data to yield the converted data.

The data processing module 124 may also comprise a data capture module 920. The data capture module 920 may include one or more hardware, software, and/or firmware elements that determine which data received by and/or temporarily stored in the data buffer module 910 is to be securely sent to the medical data cloud module 150 (via medical data transfer module 140), e.g., extracted and converted data, and may capture such data to be securely sent from the data buffer module 910. The data capture module 920 may then relay the data to be securely sent through a medical data transfer module interface 933. The medical data transfer module interface 933 may establish or confirm the presence of a secure pathway (e.g., a VPN, an SSH connection, an SFTP connection, etc.) for transmission to the medical data transfer module 140 and/or to the medical device controller 120 via the medical data transfer module 140.

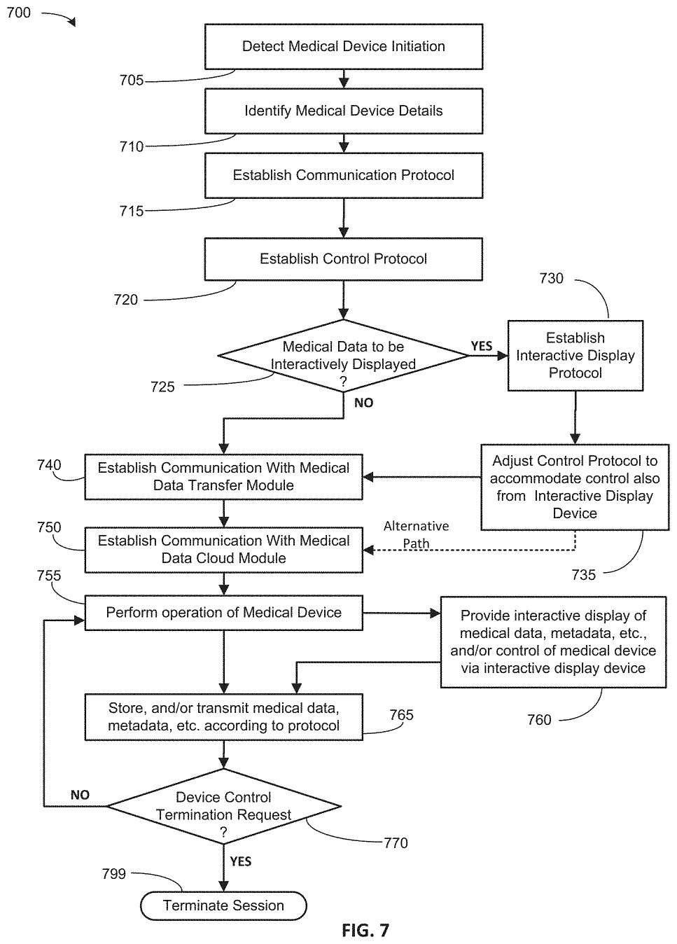

FIG. 7 presents a flowchart of a first method 700 in accordance with embodiments herein. The method 700 comprises detecting (at 705) an initiation of a medical device. For example, in the context of FIG. 1-FIG. 7, the medical device controller 120 may detect the startup of one or more medical devices 114-118, and/or the medical device controller 120 may receive an operator request to start up one or more medical devices 114-118 and may activate the medical device 114-118 in response.

In embodiments, the method 700 may also include identifying (at 710) at least one detail or parameter of the medical device, wherein the parameter relates to at least one of communication involving the medical device or control of the medical device. Identifying (at 710) may involve looking up, by medical device controller 120, of details of the medical device 114-118 that have previously been stored in database 126, querying the medical device 114-118 for such parameters, or the like.

The method 700 also comprises establishing (at 715) a communication protocol between the medical device and a data extraction module. The communication protocol may be mediated by a data routing module. For example, a communication protocol may be established (at 715) between the medical device 114-118 and the data extraction module 128 via data routing module 112. The communication protocol may be secured or unsecured, via Ethernet, Wi-Fi, Bluetooth, or two or more thereof, etc. Establishing (at 715) the protocol may also comprise establishing a protocol for communications between the data extraction module 128 and the data processing module 124, or between the medical device 114-118 and the data processing module 124 directly, if so desired and if possible.

If identifying (at 710) identified at least one parameter relating to communication involving the medical device, the communication protocol established (at 715) may be based at least in part on the identified (at 710) parameter.

The method 700 also comprises establishing (at 720) a control protocol between the medical device and a virtual machine. In the example of FIG. 1-FIG. 6, the virtual machine may be instantiated by the virtual machine module 614, such as in response to the detecting (at 705) or upon user input.

If identifying (at 710) identified at least one parameter relating to control of the medical device, the control protocol established (at 720) may be based at least in part on the identified (at 710) parameter.

In embodiments, the method 700 may further comprise determining (at 725) whether the medical data is to be displayed interactively to a user of the medical device. If the determining (at 725) is affirmative, then the method 700 may additionally comprise establishing (at 730) an interactive display protocol between the medical device and an interactive display device. The interactive display protocol may be mediated by the medical device controller 120 shown in FIG. 1 and FIG. 2, or by other implementations that will be routine to the person of ordinary skill in the art having the benefit of the present disclosure. After establishing (at 730), the method 700 may further comprise adjusting (at 735) the control protocol to accommodate partial or full control of the medical device by the interactive display device.

If the determining (at 725) is negative, flow passes to establishing (at 740 and 750) a secure communication link from the medical device through the data routing module to a medical data cloud module. In the depicted embodiment, the secure communication link is first established (at 740) from a data routing module, such as the data routing module 112, to a medical data transfer module, such as the medical data transfer module 140 shown in FIGS. 1-7, and thereafter is established (at 750) between the medical data transfer module and the medical data cloud module, such as the medical data cloud module 150 described above. One or more security protocols may be implemented in establishing the secure communication link, such as those described above. Desirably, the security protocols comply with the medical data privacy regulations of all relevant regulatory bodies, such as the U.S. FDA.

The method 700 may comprise performing (at 755) one or more operations of the medical device, e.g. capturing an ultrasound image by an ultrasound device, capturing EKG data by an EKG, etc. If medical data was determined (at 725) to be interactively displayed, the method 700 may comprise providing (at 760) the medical data to the interactive display device 130 to display and/or control. Whether or not providing (at 760) is performed, the method 700 may also comprise transmitting and storing (at 765) medical data from the medical device to the medical data cloud module over the secure communication link, wherein the medical data cloud module securely stores the medical data.

Another determination is performed (at 770) whether a device control termination request has been received, such as from the user of the medical device, the user of the interactive display device, or from a control device (such as medical device controller 120). If no such request is received, the flow of method 700 returns to performing (at 755) one or more operations of the medical device. If such a request is received, the method terminates (at 799).

wherein at least one of the establishing the communication protocol or the establishing the control protocol is based at least in part on the identified parameter.

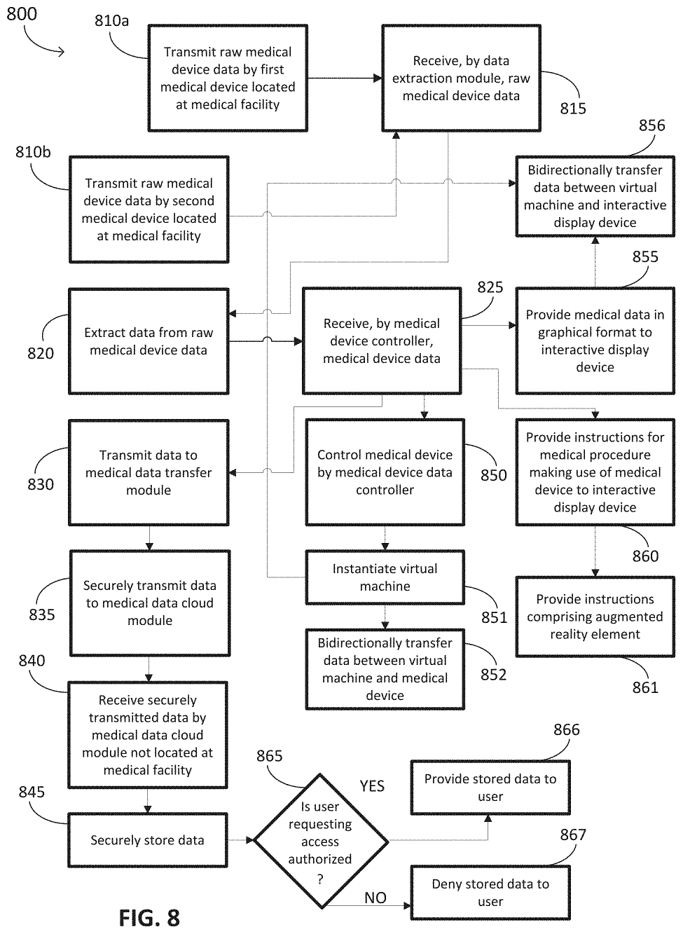

FIG. 8 presents a flowchart of a second method 8000, in accordance with embodiments herein. The method 800 begins with transmitting (at one or more blocks 810) raw medical device data by at least one medical device located at a medical facility. In the particular embodiment shown in FIG. 8, the method 800 comprises transmitting (at 810a) by a first medical device and transmitting (at 810b) by a second medical device. The number of blocks 810 may be adjusted as a routine matter by the person of ordinary skill in the art having the benefit of the present disclosure.

The method 800 may comprise receiving (at 815), by a data extraction module, the raw medical device data. The method 800 may then comprise extracting (at 820) medical device data from the medical device data by the data extraction module. The extracted medical device data may then be received (at 825) by a medical device controller.

After receiving (at 825), the method 800 may comprise transmitting (at 830) the data to a medical data transfer module. Although multiple pathways are possible, the method 800 generally comprises receiving, directly or indirectly, the medical device data by a medical data transfer module.

In embodiments, after receiving (at 825) the medical device data by the medical device controller, the method 800 may comprise providing (at 855) medical data in a graphical format to an interactive display device.

Whether or not providing (at 855) occurs, the method 800 may comprise providing (at 860) instructions for a medical procedure making use of the medical device to the interactive display device. In a particular embodiment, the method 800 may further comprise providing (at 861) instructions comprising at least one augmented reality element.

Whether or not providing (at 860) occurs, the method 800 may comprise controlling (at 850) the medical device by the medical device controller. In one embodiment, the controlling (at 850) may lead to instantiating (at 851) a virtual machine and bidirectionally transferring (at 852) data between the virtual machine and the medical device. Upon instantiating (at 851) the virtual machine, if providing (at 855) occurs, the method 800 may also comprise bidirectionally transferring (at 856) data between the virtual machine and the interactive display device.

Returning to transmitting (at 830), the method 800 may further comprise transmitting (at 835), securely, the data to a medical data cloud module. The medical data transfer module may render secure the data and/or the pathway to the medical data cloud module, although in other embodiments, other components of a system may perform one or both data security operations.

The method 800 may also comprise receiving (at 840) the securely transmitted data by a medical data cloud module, wherein the medical data cloud module is not located at the medical facility. After receiving (at 840), the method 800 may additionally comprise storing (at 845), securely, the received data by the medical data cloud module.

In embodiments, the method 800 may also include determining (at 865) whether a user requesting access to the stored data is authorized to access the stored data. In response to a determination (at 865) that the user is authorized to access the stored data, the method 800 may further comprise providing (at 866) the stored data to the user. On the other hand, in response to a determination (at 865) that the user is not authorized to access the stored data, the method 800 may comprise denying (at 867) the stored data to the user.

The particular embodiments disclosed above are illustrative only, as the invention may be modified and practiced in different but equivalent manners apparent to those skilled in the art having the benefit of the teachings herein. Examples are all intended to be non-limiting. Furthermore, exemplary details of construction or design herein shown are not intended to limit or preclude other designs achieving the same function. It is therefore evident that the particular embodiments disclosed above may be altered or modified and all such variations are considered within the scope and spirit of the invention, which are limited only by the scope of the claims.

Embodiments of the present invention disclosed and claimed herein may be made and executed without undue experimentation with the benefit of the present disclosure. While the invention has been described in terms of particular embodiments, it will be apparent to those of skill in the art that variations may be applied to systems and apparatus described herein without departing from the concept, spirit and scope of the invention.

What is claimed is:

1. A system, comprising:
at least one medical device located at a medical facility, wherein each medical device is configured to transmit raw medical device data;
a data extraction module configured to (a) receive the raw medical device data and (b) extract medical device data from the raw medical device data;
a medical data transfer module configured to (a) directly or indirectly receive the medical device data from the data extraction module and (b) securely transmit the data to a medical data cloud module; and
the medical data cloud module, wherein the medical data cloud module is not located at the medical facility, and the medical data cloud module is configured to (a) receive the data securely transmitted by the medical data transfer module and (b) securely store the received data.

2. The system of claim 1, further comprising:
a data routing module configured to (a) receive the raw medical device data transmitted by each medical device and (b) directly or indirectly transmit the raw medical device data to the data extraction module.

3. The system of claim 1, further comprising:
a medical device controller configured to (a) receive the medical device data transmitted by the data extraction module and (b) transmit the data received from each medical device to the medical data transfer module.

4. The system of claim 3, wherein the medical device controller is further configured to (c) control the at least one medical device, (d) provide the medical device data in a graphical format to an interactive display device, (e) provide instructions for a medical procedure making use of the at least one medical device to the interactive display device, or (f) two or more of (c)-(e).

5. The system of claim 4, wherein the medical device controller is configured to (c) control the at least one medical device by instantiating a virtual machine and bidirectionally transferring data between the virtual machine and the medical device.

6. The system of claim 5, wherein the medical device controller is configured to (d) provide the medical data in a graphical format to an interactive display device by bidirectionally transferring data between the virtual machine and the interactive display device.

7. The system of claim 4, wherein the medical device controller is configured to (e) provide instructions for a medical procedure making use of the at least one medical device to the interactive display device, wherein the instructions comprise at least one augmented reality element.

8. The system of claim 1, wherein the medical data cloud module is further configured to (c) securely provide the stored data to an authorized user.

9. A method, comprising:
transmitting raw medical device data by at least one medical device located at a medical facility;
extracting medical device data from the raw medical device data by a data extraction module;
transmitting, securely, the data to a medical data cloud module;
receiving the securely transmitted data by a medical data cloud module, wherein the medical data cloud module is not located at the medical facility; and
storing, securely, the received data by the medical data cloud module.

10. The method of claim 9, further comprising:
receiving, by a data routing module, the raw medical device data transmitted by each medical device; and
transmitting, directly or indirectly, the raw medical device data to the data extraction module.

11. The method of claim 9, further comprising:
receiving, by a medical device controller, the medical device data extracted by the data extraction module, and
transmitting, by the medical data controller, the medical device data to the medical data transfer module.

12. The method of claim 11, further comprising:
controlling the at least one medical device by the medical device controller,
providing the medical device data, by the medical device controller, in a graphical format to an interactive display device,
providing instructions for a medical procedure making use of the at least one medical device by the medical device controller to the interactive display device, or
two or more thereof.

13. The method of claim 12, wherein controlling the at least one medical device comprises:
instantiating, by the medical device controller, a virtual machine, and
bidirectionally transferring data between the virtual machine and the medical device.

14. The method of claim 13, wherein providing the medical device data in a graphical format to an interactive display device comprises bidirectionally transferring data between the virtual machine and the interactive display device.

15. The method of claim 12, wherein providing instructions for a medical procedure making use of the at least one medical device to the interactive display device comprises providing at least one augmented reality element.

16. The method of claim 9, further comprising:
determining whether a user requesting access to the stored data is authorized to access the stored data;
providing the stored data to the user, in response to a determination that the user is authorized to access the stored data; and
denying the stored data to the user, in response to a determination that the user is not authorized to access the stored data.

17. A method, comprising:
detecting an initiation of a medical device;
establishing a communication protocol between the medical device and a data extraction module;
establishing a control protocol between the medical device and a virtual machine;
establishing a secure communication link from the medical device through the data routing module to a medical data cloud module;
transmitting medical data from the medical device to the medical data cloud module over the secure communication link; and
storing the medical data by the medical data cloud module.

18. The method of claim 17, further comprising:
identifying at least one parameter of the medical device, wherein the parameter relates to at least one of communication involving the medical device or control of the medical device; and
wherein at least one of the establishing the communication protocol or the establishing the control protocol is based at least in part on the identified parameter.

19. The method of claim 17, further comprising:
determining whether the medical data is to be displayed interactively to a user of the medical device;
establishing an interactive display protocol between the medical device and an interactive display device; and
adjusting the control protocol to accommodate partial or full control of the medical device by the interactive display device.
